(19) United States
(12) Patent Application Publication
(10) Pub. No.: US 2015/0247797 A1
(43) Pub. Date: Sep. 3, 2015

(54) MICROCHANNEL PLASMON RESONANCE BIOSENSOR

(71) Applicant: ALFRED E. MANN FOUNDATION FOR SCIENTIFIC RESEARCH, Santa Clarita, CA (US)

(72) Inventor: Keith Oberg, Valencia, CA (US)

(73) Assignee: ALFRED E. MANN FOUNDATION FOR SCIENTIFIC RESEARCH, Santa Clarita, CA (US)

(21) Appl. No.: 14/430,196

(22) PCT Filed: Sep. 24, 2013
(86) PCT No.: PCT/US2013/061454

§ 371 Date: Mar. 20, 2015

Related U.S. Application Data
(60) Provisional application No. 61/705,548, filed on Sep. 25, 2012.

(51) Int. Cl. G01N 21/552; A61B 5/1473; A61B 5/145; A61B 5/07; A61B 5/1459; A61B 5/00

(52) U.S. Cl. CPC G01N 21/554; A61B 5/1459; A61B 5/6833; A61B 5/1451; A61B 5/14532; A61B 5/076; A61B 5/14735; A61B 2560/0219; A61B 2562/0233

(57) ABSTRACT

Methods and systems for biosensing are disclosed, based on microchannel surface plasmon resonance. A surface plasmon resonance (SPR) sensor (100) for detecting the presence of a target analyte in a fluid, comprising: a light source (105); a light transmissive substrate (112); a metal coating (114) of gold, silver or copper disposed on the substrate; a test SPR element formed in the metal coating, the test SPR element comprising: at least one test microchannel (122) in the metal coating, the at least one test microchannel having at least one aperture (124) for the passage of light from the light source through the substrate, the at least one test microchannel configured to sustain a test plasmon resonance wave, wherein the test plasmon resonance wave emits a test surface plasmon emission (SPE); and a first coating (126) in the test microchannel, the first coating comprising capture molecules selected to interact with the target analyte; a test detector (130) configured to detect the intensity of the light of the test channel (120) SPE in a predetermined wavelength band; and a reference SPR element formed in the substrate, the reference SPR element comprising: at least one reference microchannel (142) in the metal coating, the at least one reference microchannel having at least one aperture (144) for the passage of light from the light source through the substrate, the at least one reference microchannel configured to sustain a reference plasmon resonance wave, wherein the reference plasmon resonance wave emits a reference SPE, a reference detector (150) configured to detect the intensity of the light of the reference SPE in the predetermined wavelength band; and a controller (160) coupled to the test detector and the reference detector. The sensor can be implanted in a human body and can communicate and be powered wirelessly with an external coil placed in proximity to the implanted sensor or with a coil in an adhesive, external patch.

### CROSS REFERENCE TO RELATED APPLICATIONS

The present application claims priority to U.S. Provisional Patent Application No. 61/705,548, filed on Sep. 25, 2012, the disclosure of which is incorporated herein by reference in its entirety.

### TECHNICAL FIELD

The present disclosure relates to biosensors. More particularly, it relates to plasmon resonance biosensors systems and methods.

### BRIEF DESCRIPTION OF DRAWINGS

The accompanying drawings, which are incorporated into and constitute a part of this specification, illustrate one or more embodiments of the present disclosure and, together with the description of example embodiments, serve to explain the principles and implementations of the disclosure.

FIG. 1 depicts an exemplary plasmon resonance sensor.

FIG. 2 illustrates a microchannel sensor with a reference coating.

FIG. 3 illustrates a microchannel sensor with a second coating layer.

FIG. 4 illustrates a microchannel sensor with a molecule mixture in the microchannels.

FIG. 5 illustrates two exemplary geometries for light emission and detection.

FIG. 6 illustrates an exemplary system for implantation.

FIG. 7 illustrates exemplary sensor device components.

FIG. 8 illustrates exemplary external device components.

FIGS. 9A and 9B illustrate an exemplary V-shaped sensing structure in top and cross-sectional side view.

FIGS. 10A and 10B illustrate an exemplary sensing with multiple microchannels for the test and reference channels.

FIG. 11 illustrates an exemplary intensity vs. wavelength graph.

FIG. 12 illustrates an exemplary response for step increases in target analyte concentration.

### SUMMARY

In a first aspect of the disclosure, a surface plasmon resonance (SPR) sensor for detecting the presence of a target analyte in a fluid is described, the sensor comprising: a light source; a light transmissive substrate; a metal coating of gold, silver or copper disposed on the substrate; a test SPR element formed in the metal coating, the test SPR element comprising: at least one test microchannel in the metal coating, the at least one test microchannel having at least one aperture for the passage of light from the light source through the substrate, the at least one test microchannel configured to sustain a test plasmon resonance wave, wherein the test plasmon resonance wave emits a test surface plasmon emission (SPE); and a first coating in the test microchannel, the first coating comprising capture molecules selected to interact with the target analyte; a test detector configured to detect the intensity of the light of the test channel SPE in a predetermined wavelength band; and a reference SPR element formed in the substrate, the reference SPR element comprising: at least one reference microchannel in the metal coating, the at least one reference microchannel having at least one aperture for the passage of light from the light source through the substrate, the at least one reference microchannel configured to sustain a reference plasmon resonance wave, wherein the reference plasmon resonance wave emits a reference surface plasmon emission (SPE), a reference detector configured to detect the intensity of the light of the reference SPE in the predetermined wavelength band; and a controller coupled to the test detector and the reference detector.

### DETAILED DESCRIPTION

Continuous, active monitoring of biomarkers is not a routine medical practice: there is no system known to the person skilled in the art that meets the criteria for widespread adoption. Several embodiments of the present disclosure comprise a platform technology for implanted biosensors. As a consequence, it may be possible to eliminate the major obstacles to adoption and develop a system that is small, simple, reliable, inexpensive, accurate, robust, convenient, implantable and versatile.

The present disclosure describes a micro-sensor platform based on the surface plasmon resonance (SPR) effect. Surface plasmon resonance is sensitive to refractive index in a way that allows precise data to be collected from simple hardware. SPR measurements are based on the optical properties of resonant cavities smaller than a human hair, and require no enzymes or fluorescence.

Surface plasmon resonance is an effect that occurs at the surface of some metal films or nanoparticles when they are irradiated with light. Molecules near the metal surface strongly affect the nature of the resonance, so the effect can be used to measure very small changes in a sample at the metal surface. SPR is one of the most sensitive optical methods known: zeptomol sensitivity has been reported. There are several ways to configure SPR equipment, known to those skilled in the art, for the purpose of making different types of measurement. A configuration that can be optimal in some embodiments of the invention disclosed here is microchannel SPR.

In microchannel SPR, a sub-wavelength slit or hole (or pattern of such features) in a metal film is filled or covered with receptor molecules. The slit is illuminated from one side, and the light emitted from the resonant cavity is measured from the other. A change in the spectrum of the emitted light occurs when the target molecules binds to the receptors. By measuring the intensity of one or more wavelengths transmitted through the slit, a concentration estimation can be made. Since this method is so sensitive, it can respond to all changes in the interstitial fluid at the implant site, including those not caused by the target analyte. If a reference slit containing an inert protein (e.g. casein or albumin etc.) is added to the sensor, then non-analyte related signals can be subtracted from the analyte signal.

There are numerous disease conditions in which the amount of a minor constituent of body fluids changes. An example is diabetes, where the glucose levels fluctuate between 0.05-0.2%. Glucose sensing is used as an exemplary system, but this invention can be applied to many systems. Other diseases with trace markers comprise cancers, rheumatoid arthritis, thyroid conditions, liver diseases, coronary artery disease, etc. In these cases, the quantity of marker can fall below one part per billion.

Currently, measurement of disease markers requires collection of a blood sample which is then transported to a laboratory, processed by a technician and ultimately run in an immunoassay such as ELISA or RIA which is labor intensive and requires specialized operator skills, as well as typically requiring several days to complete.

Because of its simplicity and the naturally small dimensions of the plasmon-resonance microchannels, this type of sensor can be easily miniaturized and implanted. An implanted version could provide continuous monitoring for various disease states, such as cancer relapse, hepatitis, or blood sugar levels in diabetics.

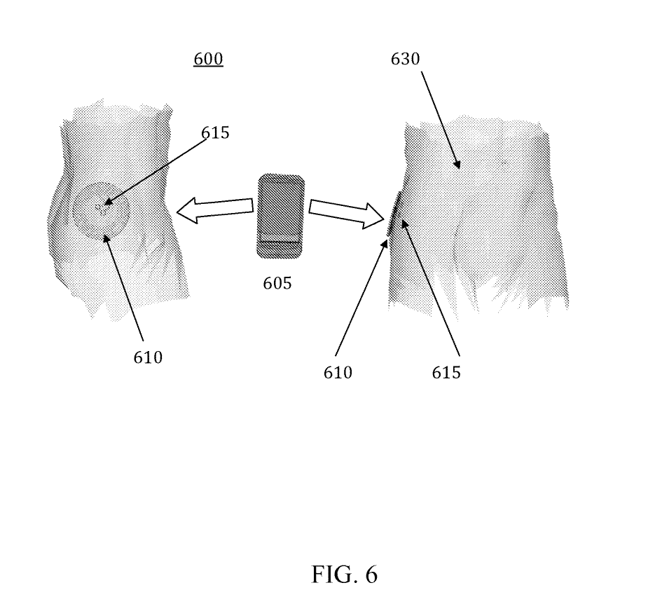

As illustrated in FIG. 6, the sensors of the present disclosure can be part of an integrated monitoring system (600) that includes a computer or handheld controller (605), an adhesive external patch (610) and implanted micro-sensor modules (615) that, in some embodiments, can, for example, measure about 3×3×10 mm. Receptor biomolecules (oligonucleotides or antibodies) near the surface of each micro-sensor can give specificity, but the internal optoelectronic components can be identical in all sensor types, making this a customizable platform technology.

The implanted micro-sensors (615), which can be implanted in biological tissue, such as for example a human body (630), can be charged through the external patch (610) or an external coil positioned in proximity to the implanted micro-sensors, and a computer or smartphone (605) can be loaded with patient monitoring software.

The implanted micro-sensor module (615) can be a biocompatible hermetic package, for example, with a total volume in the range 0.1-0.2 mL. This unit (615) can be implanted with a small trochar in an out-patient procedure. The micro-sensor (615) is completely internal, with no percutaneous elements.

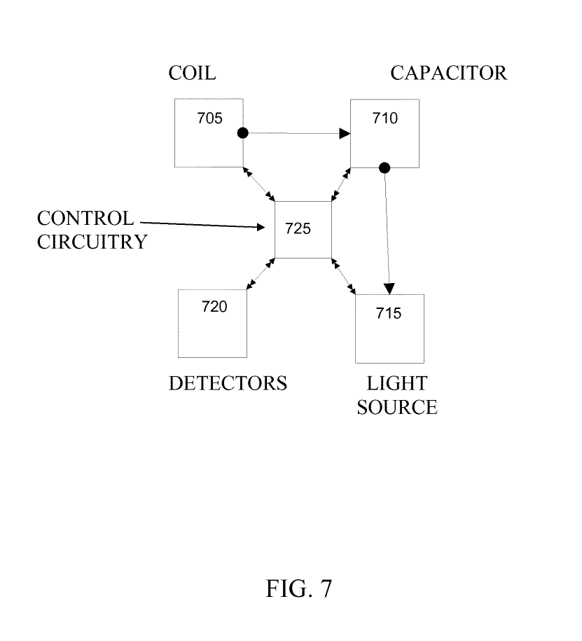

As illustrated in FIG. 7, the internal components of some embodiments of the disclosure can comprise a coil for telemetry and charging (705), a high-efficiency capacitor (710) for power storage, an HLED or laser diode light source (715), photodiodes for detection (720), and control circuitry (725). In several embodiments, the sensor can capture and transmit data, without an on-board processing or data storage capability.

The use of coils to power sensor implants is known to the person skilled in the art. For example, such systems are described in U.S. Pat. Nos 5,193,539; 5,324,316; 6,067,474; 6,208,894; 6,315,721; and 6,564,807, the disclosure of all of which is incorporated herein by reference in their entirety.

To minimize the need for calibration, the micro-sensor can have a dual-channel design with at least one test microchannel and at least one reference microchannel, as described above in the present disclosure. The microchannels can be identical except that the test microchannel comprises a capture molecule, such as antibody or a DNA oligomer, which can concentrate a target analyte in the plasmon resonance cavity. Even a small amount of target molecule binding will cause a change in transmitted light intensity due to the plasmon resonance effect. This difference can be used to calculate analyte concentration.

The external component of the system—such as patch (610) in FIG. 6—can be a soft patch with a thickness of, for example, about ⅛″, that can be held on the skin above the implanted sensor by an adhesive. The external component can be a waterproof unit made of a flexible material, for example with a look and feel similar to a nicotine patch. In other embodiments, the external component may not be an adhesive patch, but other suitable replacement, as understood by the person skilled in the art.

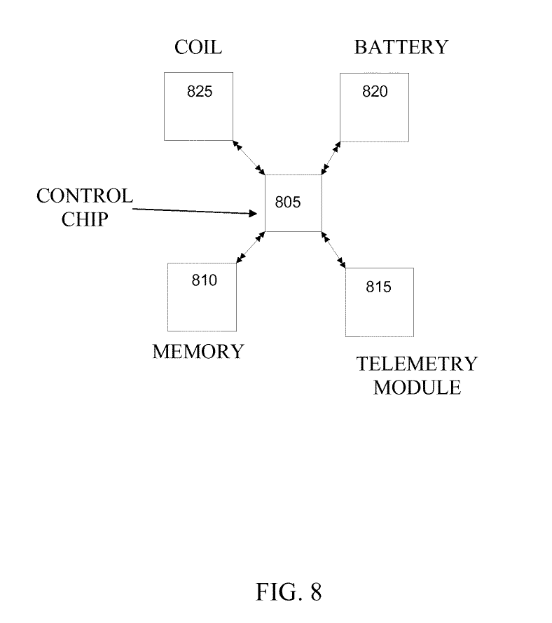

As illustrated in FIG. 8, the external component can comprise a control chip (805), memory (810), a telemetry module (815), a battery (820), and an RF coil (825) that powers and communicates with the implanted micro-sensor. The external component can collect and store data from the sensor, for upload to a separate controller module (605). In other embodiments some or all of these components can be contained within the implant together with the plasmon resonance components, as understood by the person skilled in the art.

The controller module (605) can vary depending on the application. In some embodiments, the controller module can be a patient module (e.g. for glucose monitoring), or a physician module for other biomarkers. These modules can be used to collect and analyze data from the sensor as well as to program the behavior of the implanted sensor as well as of the external component, such as an adhesive patch or wireless power transfer coil. In some embodiments, the controller module is implemented through controller software in smart-phones or tablet computers using telemetry such as Bluetooth communications.

As known to the person skilled in the art, recent fabrication of microchannel plasmon resonance (SPR) structures has made possible short path length applications, and therefore miniaturization. In microchannel plasmon resonance the intensity of light transmitted through a resonant cavity is measured. The resonant cavity can be exceedingly small, of the order of 0.3 micron deep×1 micron wide×15 microns long, and the intensity profile of transmitted light can be highly sensitive to the material in the cavity. Some demonstrations of microchannel plasmon resonance being used for biomolecule detection comprise: Leebeck et al. Annal. Chem., 2007; Amarie et al. Annal. Chem., 2010; Lepage et al. Nano. Res. Lett. 2011; and Amarie et al. Annal. Chem., 2010, the disclosure of all of which is incorporated herein by reference in their entirety. In particular, Amarie et al. 2010 reported that as little as one zeptomole of analyte could be detected.

The high sensitivity of microchannel plasmon resonance is well suited to biomarker monitoring in humans. A sensor built around microchannel plasmon resonance can function with similar specificity to ELISA, can allow frequent sampling, and can be minimally invasive. In some embodiments, it can require only subcutaneous insertion of a 3×3×10 mm implant and the use of an external RF control unit.

In several embodiments, the sensor device is intended to be bathed in a body fluid such as blood or interstitial fluid. Long-term implantation of such a device requires a hermetic capsule to house the optoelectronic components that provide the photostimulation of the plasmon resonance, and collect its resulting photoemission. Short-term implantation could be done without a hermetically sealed capsule.

Several embodiments of the present disclosure comprise at least two microchannel plasmon resonance chambers that have been micromachined or formed into a metal coating (for example, made of gold) on a transparent substrate (for example, made of glass).

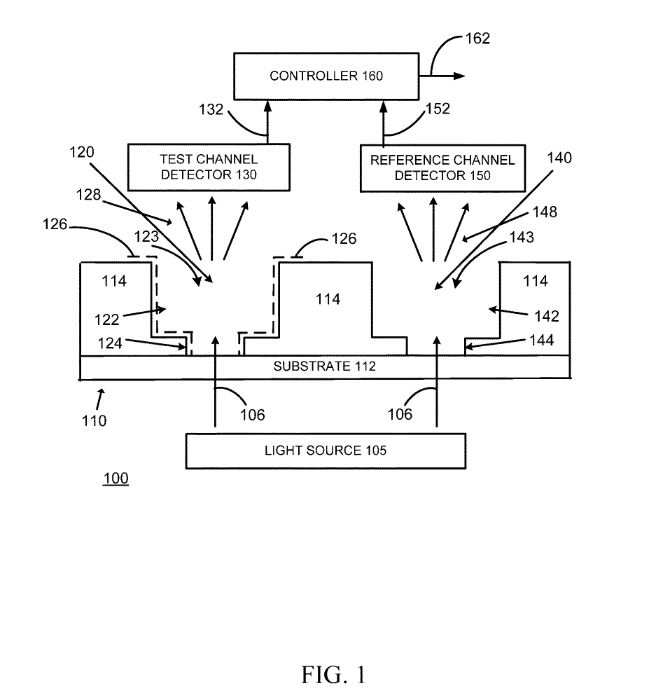

FIG. 1 illustrates one embodiment of a microchannel plasmon resonance sensor (100), where a test channel (120) including at least one test microchannel (122), is employed for test or sensing and a reference channel (140) including at least one reference microchannel (142) is employed as a reference. Light source (105) shines light (106) through transparent substrate (112), which is covered with metal coating (114). Apertures or slits (124, 144), test microchannel (122) and reference microchannel (142) are formed or molded into metal layer (114). Test microchannel (122) includes a coating of capture molecules (126). When light (106) shines through slit (124), then within test chamber (123) in test microchannel (122), a plasmon resonance wave is formed and light (128) is emitted toward test channel detector (130). Similarly, when light (106) shines through slit (144), then within reference chamber (143) in reference microchannel (142), a plasmon resonance wave is formed and light (148) is emitted toward test channel detector (150). Detectors (130, 150) are photodetectors, with respective output signals (132, 152) coupled to controller (160) for processing of those signals and for generating an output (162) which is coupled to other circuits or systems as needed for a particular sensing application. The aperture or slit (124, 144) would generally be of sub-wavelength dimension in terms of width.

Test microchannel (122), is filled or lined with a coating of a capture or receptor molecule (126) that will interact or bind to a target of interest. Examples of receptor molecules comprise antibodies, cellular receptor molecules, restriction endonuclease, phenylboronic acid derivatives, polynucleic acids, and components of a multiprotein complex. Examples of targets of interest comprise antigens, proteins, RNA sequences, DNA sequences, small molecules, sugar molecules, and nutrients.

The control or reference channel (140) includes at least one reference microchannel (142), and is treated in a manner identical to the test microchannel (122), except that such a bare reference microchannel (142) as shown in FIG. 1 does not preferentially bind to the target molecule,

The test microchannel (120) and control channel (140) are illuminated from below. Light (106) from the source (105) passes through an aperture or slit (124, 144) in the bottom of each microchannel (120, 140) and resonates in each microchannel (120, 140). The aperture or slit (124, 144) would generally be of sub-wavelength dimension in terms of width.

A biological fluid to be tested (e.g. blood) is washed over the top of the microchannels (122, 142). Pre-filters, protective hydrogel layers or other methods may be used to block cells from the blood and prevent them from entering the light path. The target molecule, if present, will diffuse into all microchannels (122, 142) in the sensor (100). Because of the small dimensions of the microchannels, diffusion will be rapid. In the test microchannel (122) in FIG. 1, the target will interact with or be reversibly bound by the binding molecules and additional target will diffuse into the microchannel, thus creating a higher concentration of target molecules in the test microchannel than exists in the reference microchannel (140) in FIG. 1. This will cause a refractive index difference between the test and reference microchannels—such as (122) and (142) in FIG. 1.

Depending on the total refractive index of all materials in each microchannel (120, 140), the light emitted (128, 148) will vary in intensity. The ratio or difference in intensities between the test signal (128) and the reference signal (148) will be related to the target analyte concentration in the sample being analyzed.

The sensor base (110) which supports the microchannels (122, 142) is built on a substrate (112). An example of light source (105) would be a LED or laser diode light source.

In several embodiments, the microchannels could be milled or molded into a metal coating. The same light source can be used for all microchannels, to ensure consistency and stability of the signals. The person skilled in the art will understand that a different number of microchannels could be used, for both test and reference channels, and that different geometries could be used to align the light source with the microchannels and detectors.

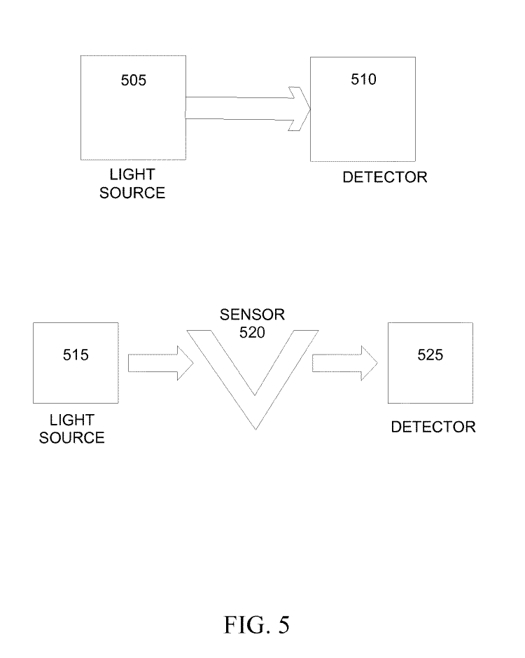

For example, referring to FIG. 5, a light source (505) could be present on one side, with the detectors (510) on the opposite side. Alternatively, the light source (515) and detectors (525) could be emitting and receiving light at an angle, with the microchannel device (520) fabricated in a V-shaped groove.

The addition of target molecules into a sensor device according to some embodiments of the present disclosure can cause intensity changes in different parts of the spectrum of the transmitted light. For example a step increase in glucose concentration would produce stepwise changes in the SPE intensity at both the test and reference channels, and a corresponding stepwise increase in the difference between them. The signal change may be maximal in specific wavelength bands or multiple wavelength bands. Determination of the concentration of an analyte in a fluid can be performed by a comparative analysis of the light emitted from the test and reference microchannels of an SPR sensor according to the present invention.

In some embodiments, more than one wavelength may be used for the light source, with an increase in measurement accuracy.

Although light is used for detection, said light is not necessarily absorbed by any material or molecules within the test or reference microchannels. Instead, the resonant frequency and/or resonant quality factor of the chamber will be affected by the biological content or materials within the chamber. Therefore it is possible to realize completely passive receptor systems. An advantage of using passive receptor systems would be that signal degradation relating to the photo-bleaching that often occurs in fluorescence detection systems and/or the chemically reactive byproducts generated in enzymatic detection systems would not be problematic.

Advantages of several embodiments of the present disclosure comprise a high sensitivity. In fact a sensitivity for glucose concentrations as small as 100 mg/dL=1 g/L=0.1% (w/v) may be achieved and sensitivity lower than 1 μg/L (1 PPB) is possible in some other applications as described above in this disclosure. Such a small signal is difficult to measure directly with many analytical techniques.

In some applications, the signal from glucose must be uniquely identified (specificity). In such applications, there must be no or negligible signal from any and all other components that may be in a sample being measured. In such cases, the signal must not be affected by changes in the matrix and must be proportional to the glucose concentration in the blood.

In some glucose sensor embodiments, the response-delay of the sensor (kinetics) can be short enough that closed loop control is possible. This can be true for both rising and falling glucose concentration. As would be understood by one skilled in the art, the lag time of a glucose sensor can be caused by a combination of 1) any inherent lag in the sensor (i.e. glucose diffusing into the active sensor element) and 2) the lag between changes in blood glucose and the glucose level in the fluid surrounding the sensor (for a sensor implanted in tissue). One strategy to minimizing 1) is by making the physical diffusion barriers in the sensor as thin as possible. Because of the sub-micron depth of the microchannels in this sensor, diffusion of analyte through protective hydrogel layers (327, 347, 434, 454) will be rapid.

In several embodiments, an extended lifetime can be expected because capture reagents used for glucose detection (e.g. GOX or phenylboronate molecules) can be protected from degradation by the design of the coatings inside the microcavities and on the exterior surfaces of the device. Several complementary strategies to maximizing longevity can be used. They comprise strategies for evading immune response such as a steroid-eluting erodible coating and a long-term stealth coating of long-chain polyethylene glycol or other biocompatible molecules, as would be known to one skilled in the art. They also comprise strategies to maximize the ruggedness of the sensing element, such as chemically bonding the active sensing molecules to the detector surface, coating the capture molecules with a protective layer, and using capture molecules that are resistant to chemical degradation.

In the present disclosure, amplification and/or specificity of the sensors comprise a method of increasing the concentration of target molecule, glucose for example, at the test element—the test microchannel. Numerous different glucose binding molecules could accomplish this task such as glucose receptor, glucose binding proteins, glucose dehydrogenase, glucose oxidase, or phenylboronic acid derivatives. Suitable molecules can be chosen based on how well they meet the requirements for sensitivity, specificity, kinetics and lifetime. The matrix may comprise capture molecules crosslinked directly, with spacer-crosslinkers, embedded in a polymeric matrix or embedded in a matrix of crosslinked protein.

In the case of glucose sensing, using molecules that bind to glucose with high affinity (and bind to none or only a few other molecules), as well as coating the active surface of the sensor with these molecules, will increase the glucose signal relative to the other components in the fluid being sampled. By choosing a molecule that binds and releases glucose on a relatively fast timescale, the concentration of glucose at the sample channel can be proportional to the bulk concentration as described by the glucose binding equilibrium constant of the binding molecule.

Another possible way to enhance the specificity of analyte concentration measurement comprises using a reference channel that has identical conditions to the sample channel except for the analyte concentration. Again, using glucose as an example, the signal from a reference channel allows correction for any background signal that may be generated by non-glucose effects at the sensor head. To be effective, the reference channel can have the same design as the main channel. The only difference can be that the reference is loaded with a non-glucose binding molecule that is similar in properties to the glucose binding molecule in the sample channel. In this way, the signals from both test and reference channel can be matched, except for the signal that is due to glucose itself. Subtracting, ratioing, or otherwise comparing the two channels will produce a difference signal that is related to excess glucose concentration at the sample sensor surface. This method can compensate for variation in factors such as pH, temperature, osmolarity, electrolytes, drugs, urea, ketosis, metabolic disorders, etc. which may cause changes in signal intensity in both the test and reference channels.

One example of the many different molecules that could be used for amplifying glucose concentration in the sensor region is glucose oxidase (GOX), a good candidate because it can be incorporated in a robust matrix that can remain stable for a planned lifetime of the sensor. One potential disadvantage of natural GOX is that as it interacts with glucose, hydrogen peroxide is generated, which can attack the GOX that produced it and cause loss of sensitivity. A potential solution to this problem is to eliminate the enzymatic activity of GOX while preserving its glucose-binding activity. GOX modified in this way will still increase the local glucose concentration at the sensor surface, thus enabling the sensitive detection of glucose and the determination of its concentration. It is also possible to adjust the strength with which GOX binds to glucose, so that the kinetics and concentration enhancement can be adjusted to give optimal sensitivity and response time. The active site residues of GOX have been studied and mutants or synthetic versions that have a wide variety of kinetic parameters and binding properties are known to the person skilled in the art.

Other exemplary glucose-binding molecules comprise non-fluorescent di(phenylboronic acid) molecules with appropriate molecular structure to bind glucose with an equilibrium constant less than around 50 mM. The biggest advantage of such molecules is that they are much smaller than GOX and so could theoretically be loaded on the sensor head at a much greater density than GOX.

Some features of several embodiments of the present disclosure comprise a simple source-sample-detector geometry. In some embodiments, the system comprises a capacitor, LED source, test chamber, photodiode detector, control logic circuit and a coil for power and data transmission. Such a configuration could fit in a small package suitable for implantation in biological tissues. Another feature of several embodiments is an incident beam from a single source—the ratio of intensities will be independent of variations in the source intensity. Yet another feature of several embodiments is side-by-side test and reference channels—all changes in environment, matrix, or analyte concentration will affect both chambers simultaneously. Yet another feature of several embodiments is wavelength selection through LED emission wavelength—bandwidth may be narrow or wide as long as the intensity is modulated by the target analyte. Yet another feature of several embodiments is a simple measurement of emitted light intensity.

An advantage of several embodiments of the present disclosure is the use of crosslinked proteins in the microchannels. In typical surface plasmon resonance devices, the resonant field intensity falls off exponentially with distance from the surface. Generally, most of the energy is found within a few tens of nanometers from the detector surface. As a result, as it is known to the person skilled in the art, plasmon resonance-based sensors generally use only a monolayer of capture molecules that is immobilized (via a chemical tether) directly on a gold surface. There is not a large advantage to adding more layers of capture molecules on top of the first layer because they will contribute exponentially less to sensitivity, as a function of distance from the gold surface.

With the microchannel design of the present disclosure, most of the volume inside the channel can participate in the resonance effect. Therefore, filling the channel with multiple layers of receptor molecule can lead to a higher sensitivity per unit area than can be achieved with monolayer sensors known in the art.

An example of a possible sensor configuration that incorporates the above elements can comprise a microchannel surface plasmon resonance chamber that is filled with (enzymatically inactivated) glucose oxidase in the test microchannel and a similar amount of inert protein in the reference microchannel. A beam from a light source is split and passed through both microchannels. Detectors measure the light intensity difference from the two microchannels at a wavelength that is sensitive to analyte concentration.

In addition, covalently incorporating the receptor molecule in a crosslinked hydrogel could have significant implications to the working lifetime of the sensor. The receptor molecules in a monolayer are exposed to the external environment. This can mean rapid deactivation in vivo by the foreign body response. In several embodiments of the present disclosure, the capture molecules are incorporated in a hydrogel which will shield them from the external environment while not contributing (or not contributing to a significant degree) to the actual signals of interest. The hydrogel can also have the additional effect of physically stabilizing the capture molecules against denaturation or other inactivation mechanisms.

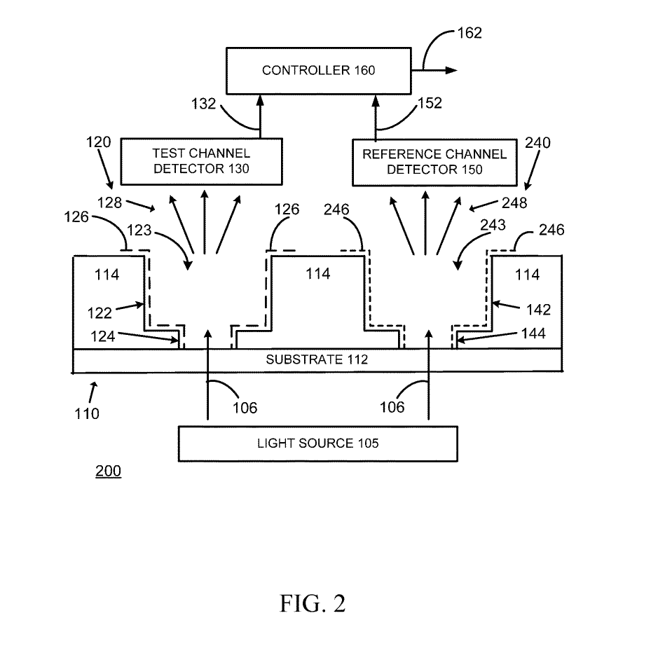

FIG. 2 illustrates an embodiment of the present disclosure, where the elements numbered similarly to FIG. 1 retain the same significance. Referring to FIG. 2, a sensor device (200) is described, comprising a reference channel (240) including at least one reference channel (143), which is coated with a coating (246) of inert molecules that does not preferentially bind to the target molecule. A surface plasmon wave in reference chamber (243) in reference microchannel (143) emits a surface plasmon emission (248) which is detected by reference channel detector (150). In other embodiments, several microchannels could be used within each channel.

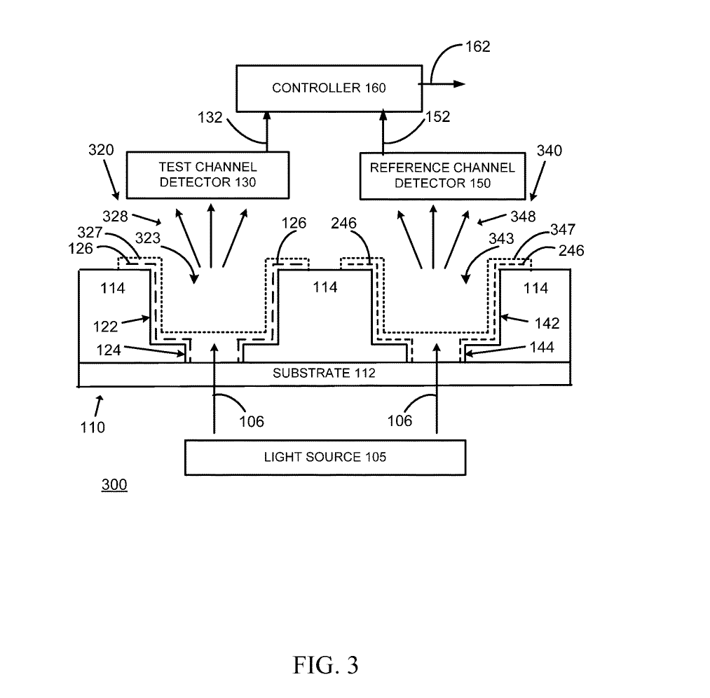

FIG. 3 illustrates an embodiment of the present disclosure, where the elements numbered similarly to FIGS. 1 and 2 retain the same significance. Referring to FIG. 3, a sensor device (300) is described, comprising a test channel (320), with at least one test microchannel (122), and a second coating (327) such as a hydrogel. The hydrogel can shield the capture molecules (126) in the first coating from the external environment while not contributing to any significant degree to the actual signals of interest. The hydrogel can also have the additional effect of physically stabilizing the capture molecules against denaturation or other inactivation mechanisms. A surface plasmon wave in test chamber (323) in test microchannel (122) emits a surface plasmon emission (328) which is detected by test channel detector (130). Sensor (300) also comprises reference channel (340) including at least one reference microchannel (142), which also has a second or protective coating (347) on top of the first inert coating (246). The emission (348) from the reference microchannel (142) is detected by detector (150). Receptor molecule (126) can be immobilized by docking via a crosslinker to the surface of test microchannel (122) or by being embedded in a hydrogel.

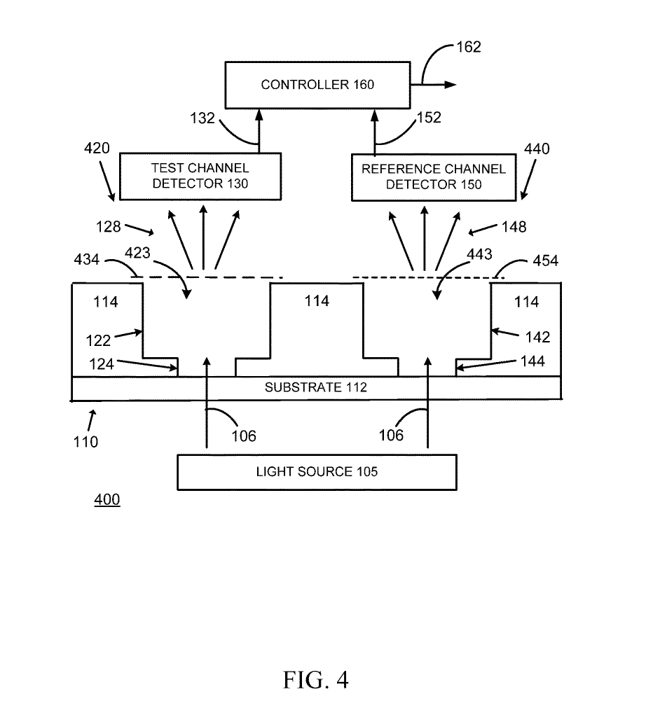

FIG. 4 illustrates an embodiment of the present disclosure, where the elements numbered similarly to FIG. 1 retain the same significance. Referring to FIG. 4, a sensor device (400) is described, comprising a test channel (420) including at least one test microchannel (122), a test chamber (423) and a mixture (434) contained in test chamber (423). The mixture may be a mixture of components used to make the test coatings described in the present disclosure, for example the coatings (126) and (327) of FIG. 3. The mixture may be a porous matrix, for example a porous matrix with capture molecules, possibly mixed, for example, with a hydrogel. The matrix may be porous to be permeable to a target analyte. As understood by the person skilled in the art, the mixture in a reference chamber would not comprise capture molecules, but inert molecules. In some embodiments, the mixture in a test chamber, such as (423), may be a mixture of capture molecules and filler molecules, while the mixture in the reference chamber, such as (443), may be a mixture of inert molecules and filler molecules. Referring again to FIG. 4, sensor (400) also comprises a reference channel (440) including at least one reference microchannel (142), with a reference chamber (443) and a mixture (454) contained in chamber (443).

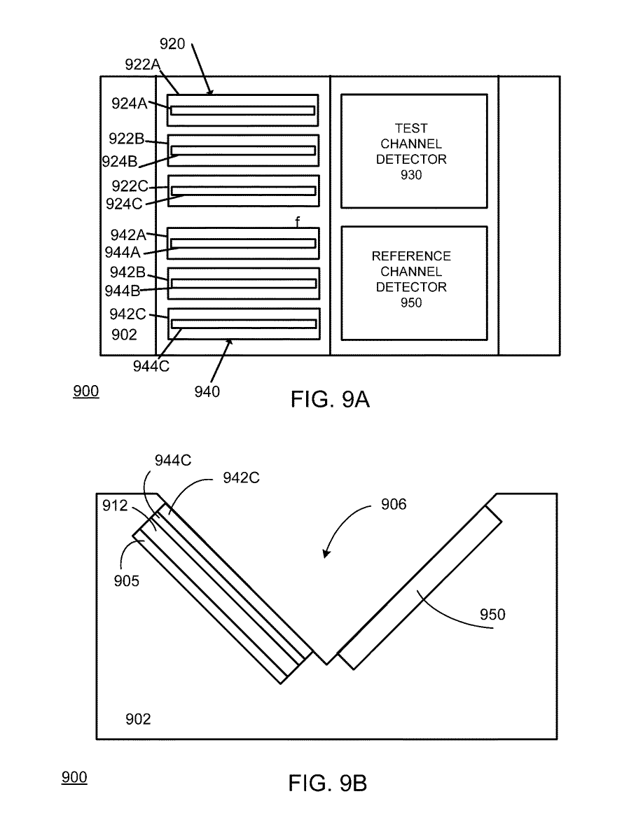

Referring now to FIG. 5, in one embodiment (520) the sensor is fabricated in a V-shaped structure. One example of this embodiment is detailed in FIGS. 9A and 9B, where FIG. 9A is a top view of a structure (900), and FIG. 9B is a cross sectional side view of the same structure (900). Other shapes could be used instead of a V shape, for example a U shape.

Referring to FIGS. 9A and 9B, a SPR sensor (900) is described, supported by a base structure (902), and comprising a light source (905). A space (906) on the outer surface of the device is available for diffusion of test fluids which may contain a target analyte. The light source (905) may be directed to illuminate a substrate (912). The device (900) comprises a test channel for sensing (920), which may comprise several microchannels, and a reference channel (940), which also may comprise several microchannels.

In one embodiment, three test microchannels (922A, 922B, 922C) are available, each with a corresponding aperture or slit (924A, 924B, 924C) for light transmission. Three reference microchannels (942A, 942B, 942C) are available, each with a corresponding aperture or slit (944A, 944B, 944C) for light transmission. A test channel detector (930) and reference channel detector (950) are also part of device (900).

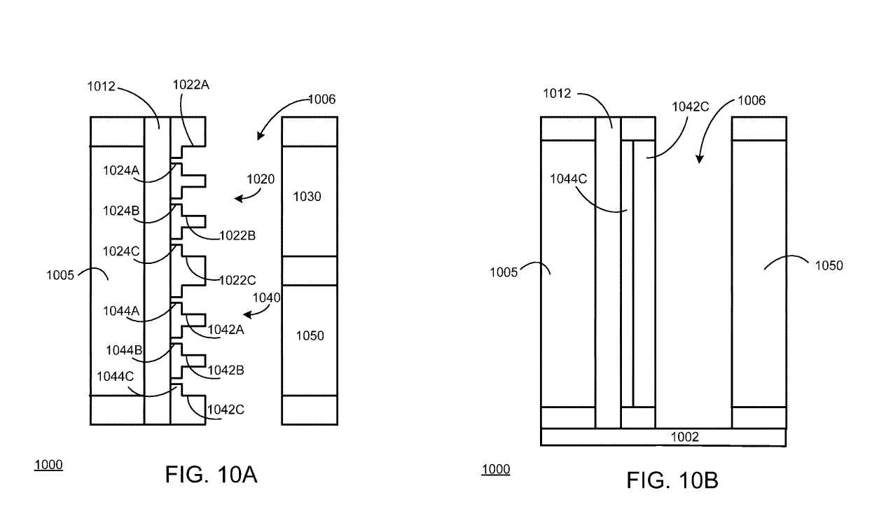

In other embodiments, the SPR sensor is not in a V-shaped structure, but still comprises multiple microchannels. For example, FIGS. 10A and 10B illustrate, respectively, a cross-sectional view and a top view of a SPR sensor.

Referring to FIGS. 10A and 10B, a SPR sensor (1000) is described, supported by a base structure (1002), and comprising a light source (1005). A space (1006) on the outer surface of the device is available for diffusion of test fluids which may contain a target analyte. The light source (1005) may be adjacent a substrate (1012). The device (1000) comprises a test channel for sensing (1020), which may comprise several microchannels, and a reference channel (1040), which also may comprise several microchannels.

In one embodiment, three test microchannels (1022A, 1022B, 1022C) are available, each with a corresponding aperture or slit (1024A, 1024B, 1024C) for light transmission. Three reference microchannels (1042A, 1042B, 1042C) are available, each with a corresponding aperture or slit (1044A, 1044B, 1044C) for light transmission. A test channel detector (1030) and reference channel detector (1050) are also part of device (1000).

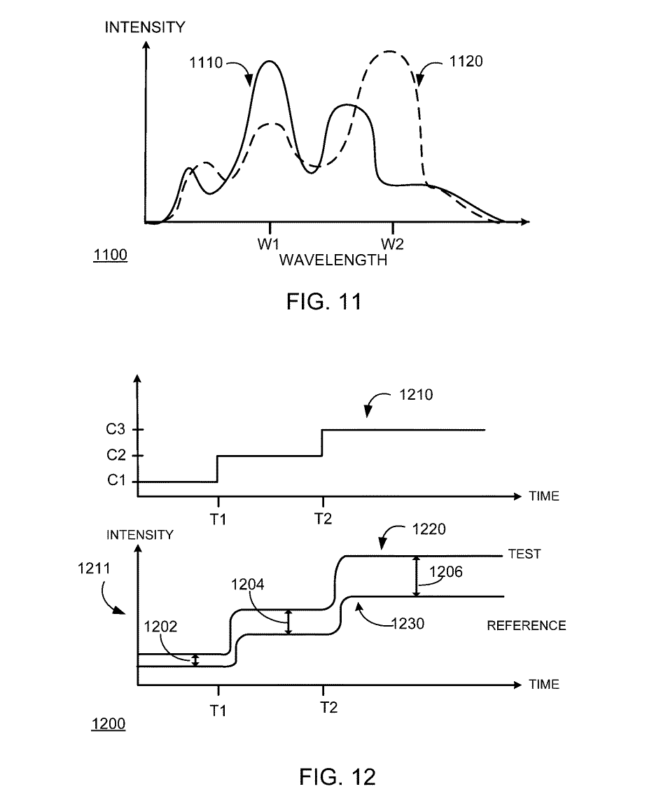

FIG. 11 illustrates an example of an intensity vs. wavelength spectrum (1100) that may be expected from several embodiments of the sensing devices of the present disclosure. The addition of target molecules into the system will cause intensity changes in different parts of the transmitted light spectrum. For example, target molecules may be introduced in space (906) of FIG. 9B. A certain intensity spectrum (1110) may be emitted at a first concentration of a target analyte, while a different intensity spectrum (1120) may be emitted at a second concentration of a target analyte. Referring again to FIG. 11, a SPR sensor, as described in several embodiments of the present disclosure, will measure an intensity value for the light due to surface plasmon resonance, at a particular wavelength or at several wavelengths. In this example, preferred wavelength bands for detecting changes in analyte concentration are centered around W1 and W2. At these wavelengths the change in emitted light intensity is largest as a function of analyte concentration. At wavelength W1 the emission intensity is maximal when analyte concentration is low (1110) and is attenuated at higher analyte concentration (1120). At W2 emission intensity increases with analyte concentration, and is maximal when analyte concentration is high (1120).

FIG. 12 illustrates two related exemplary graphs (1200) of a target analyte concentration vs. time (1210) as well as an exemplary response from the sensor (1211) that may be measured at a selected wavelength, for example W2 in FIG. 11. Referring to FIG. 12, such response (1211) comprises a measurement from the test channel as well as from the reference channel, as described in several embodiments of the present disclosure.

Referring to FIG. 12, the target analyte concentration vs. time (1210) progressively increases in a step-like fashion, going through three increasing values of concentration (C1, C2, C3). The values of concentration (C2, C3) occur at successive times (T1, T2), respectively.

Correspondingly, as the person skilled in the art will readily understand, there is a difference (1202) in response between the test channel (1220) and the reference channel (1230) of an exemplary SPR sensor, at a first value of concentration (C1). The intensity value shown is at a single wavelength, and is meant as an example, as the person skilled in the art will understand. As the concentration value increases (C2), a greater difference (1204) is noticeable between the test channel (1220) and the reference channel (1230) of an exemplary SPR sensor.

At an even higher concentration value (C3), an even greater difference (1206) will be noticeable between the test channel response (1220) and the reference channel response (1230) of an exemplary SPR sensor.

For example, the changes in target analyte concentration illustrated in FIG. 12 may refer to a step glucose concentration. Determination of the concentration of the analyte is possible by a comparative analysis of the light emitted by the test and reference channels of a sensor of the present invention.

In several embodiments of the present disclosure, capture molecules are chosen to optimize the response of a SPR sensor, based on the target analyte of interest, as well as the expected concentration of the target analyte. For example, to detect glucose concentration in a certain range, a specific capture molecule may be chosen, while for a different expected glucose concentration, a different capture molecule may be chosen.

In other words, the properties of a capture molecule may be optimized based on the type of target molecule and its expected concentration.

For such optimization, it is useful to utilize the concept of binding constant.

A binding constant may be defined as the concentration of analyte at which 50% of the capture molecules will be bound to the analyte. Mathematically, the binding constant is the point at which the rate of change of a SPR sensor signal is largest as a function of changing analyte concentration. In other words, at the derivative of the sensor signal with respect to the analyte concentration has a maximum. If the sensor signal is termed as y and the analyte concentration is termed as s, then dy/ds has a maximum at the value of the concentration analyte equal to the binding constant.

The further away the target analyte concentration is from this value, the smaller the derivative dy/ds becomes, and the more difficult it is to measure a change in target analyte. Therefore, a SPR sensor will be more sensitive at values of target analyte concentrations close to the binding constant. To optimize a SPR sensor, the binding constant should then be designed to be close to the expected value of target analyte concentration.

An exemplary working range of most molecules will be plus or minus 10 times the binding constant, although plus or minus 100 times (or greater) could be possible in some embodiments.

For example, the person skilled in the art will know that the normal blood glucose level in humans is around 5 mM. Therefore, in some embodiments, the working range of a sensor with a capture molecule binding constant of 5 mM would be roughly 0.5-50 mM. In other embodiments, for a 50 mM binding constant, the range would be roughly 5-500 mM.

A number of embodiments of the disclosure have been described. Nevertheless, it will be understood that various modifications may be made without departing from the spirit and scope of the present disclosure. Accordingly, other embodiments are within the scope of the following claims.

The examples set forth above are provided to those of ordinary skill in the art a complete disclosure and description of how to make and use the embodiments of the gamut mapping of the disclosure, and are not intended to limit the scope of what the inventor/inventors regard as their disclosure.

Modifications of the above-described modes for carrying out the methods and systems herein disclosed that are obvious to persons of skill in the art are intended to be within the scope of the following claims. All patents and publications mentioned in the specification are indicative of the levels of skill of those skilled in the art to which the disclosure pertains. All references cited in this disclosure are incorporated by reference to the same extent as if each reference had been incorporated in its entirety individually.

It is to be understood that the disclosure is not limited to particular methods or systems, which can, of course, vary. It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

1. A surface plasmon resonance (SPR) sensor for detecting the presence of a target analyte in a fluid, the sensor comprising:
a light source;
a light transmissive substrate;
a metal coating of gold, silver or copper disposed on the substrate;
a test SPR element formed in the metal coating, the test SPR element comprising:
at least one test microchannel in the metal coating, the at least one test microchannel having at least one aperture for the passage of light from the light source through the substrate, the at least one test microchannel configured to sustain a test plasmon resonance wave, wherein the test plasmon resonance wave emits a test surface plasmon emission (SPE); and
a first coating in the test microchannel, the first coating comprising capture molecules selected to interact with the target analyte;
a test detector configured to detect the intensity of the light of the test channel SPE in a predetermined wavelength band; and
a reference SPR element formed in the substrate, the reference SPR element comprising:
at least one reference microchannel in the metal coating, the at least one reference microchannel having at least one aperture for the passage of light from the light source through the substrate, the at least one reference microchannel configured to sustain a reference plasmon resonance wave, wherein the reference plasmon resonance wave emits a reference surface plasmon emission (SPE),
a reference detector configured to detect the intensity of the light of the reference SPE in the predetermined wavelength band; and
a controller coupled to the test detector and the reference detector.

2. The sensor of claim 1, wherein the controller determines the ratio of:
the intensity of the light detected by the test detector and
the intensity of the light detected by the reference detector.

3. The sensor of claim 2, wherein the controller is configured to determine a concentration of the target analyte in the fluid.

4. The sensor of claim 1, wherein the first coating in the test microchannel further comprises a crosslinker for immobilizing the capture molecules to the at least one test microchannel.

5. The sensor of claim 1 further comprising a first coating in the reference microchannel, the first coating comprising inert molecules selected not to interact with the target analyte.

6. The sensor of claim 5, wherein the first coating in the reference microchannel further comprises a crosslinker for immobilizing the inert molecules to the at least one reference microchannel.

7. The sensor of claim 5 further comprising a second coating in the test microchannel; and a second coating in the reference microchannel.

8. The sensor of claim 7, wherein the second coating in the test microchannel immobilizes the molecules of the first coating in the test microchannel; and the second coating in the reference microchannel immobilizes the molecules of the first coating in the reference microchannel.

9. The sensor of claim 5, wherein the second coating in the test microchannel comprises a hydrogel and the second coating in the reference microchannel comprises a hydrogel.

10. The sensor of claim 5 wherein the first and second coating in the test microchannel are mixed to form a porous matrix to fill the test microchannel; and the first and second coating in the reference microchannel are mixed to form a porous matrix to fill the reference microchannel.

11. The sensor of claim 1, wherein the at least one test microchannel and the at least one reference microchannel each comprise at least one microchannel with a width of about 1 micron, a length of at least 2 microns and a depth of about 100 nanometers.

12. The sensor of claim 1, wherein the target analyte is a biomarker in a fluid comprising human or animal blood, interstitial fluid, urine, sputum or mucus.

13. The sensor of claim 1, wherein the target analyte is glucose in a fluid comprising human or animal blood or interstitial fluid.

14. The sensor of claim 12, wherein the first coating in the at least one test microchannel is selected from the group consisting of: an antibody, cellular receptor molecules, restriction endonuclease, lectin, DNA, DNA analog or component of a multiprotein complex, a natural or synthetic enzyme, a catalytically inactivated enzyme, glucose oxidase, catalytically inactivated glucose oxidase, phenylboronic acid derivative and non-fluorescent phenylboronic acid derivative.

15. The sensor of claim 1 further comprising a communications system coupled to the controller for wireless communications to an external controller or implantable infusion pump.

16. The sensor of claim 1 further comprising a wireless power transfer system for powering the sensor from an external power source.

17. The sensor of claim 1, wherein the sensor is implantable in a human or animal body.

18. The sensor of claim 1, wherein the sensor is hermetically sealed.

19. A system comprising:
the sensor of claim 1;
a wireless communication and power transfer device;
wherein the wireless communication and power transfer device is coupled to the sensor.

20. The system of claim 19, wherein the communication and power transfer device comprises an adhesive patch configured to adhere to a biological surface.

21. The system of claim 20, wherein the biological surface is human skin.

22. The system of claim 19, wherein the communication and power transfer device is coupled to a computer and configured to receive software instructions, thereby allowing providing instructions to the controller.

23. The sensor of claim 1, further comprising a coil configured for telemetry and charging, a high-efficiency capacitor configured for power storage, and a control circuitry, and wherein the light source is an LED light source, and the detectors are photodiodes.

24. The system of claim 19, wherein the communication and power transfer device comprises a control chip, a memory chip, a telemetry module, a battery, and an RF coil configured to communicate and power the sensor.

25. The sensor of claim 13, wherein the at least one aperture in the at least one test microchannel and the at least one aperture in the at least one reference microchannel each have a maximum width less than a set wavelength.

26. The sensor of claim 25, wherein the at least one aperture in the at least one test microchannel and the at least one aperture in the at least one reference microchannel each have a maximum width of 10-500 nanometers.

27. The sensor of claim 25, wherein the at least one aperture in the at least one test microchannel and the at least one aperture in the at least one reference microchannel each have a maximum width of 100-300 nanometers.

28. The sensor of claim 1, wherein the target analyte is selected from the group consisting of: an antigen, a protein, a DNA sequence, a RNA sequence, small molecules, sugar molecules, biomolecules, biomarkers and a nutrient.

29. The sensor of claim 1, wherein a binding constant of the capture molecules is a function of an expected target analyte concentration.

30. The sensor of claim 29, wherein the binding constant is less than ten times and more than one tenth of the expected target analyte concentration.

31. The sensor of claim 29, wherein the binding constant is less than a hundred times and more than one hundredth of the expected target analyte concentration.

32. A method comprising:
providing the sensor of claim 1;
providing a solution to be analyzed by the sensor;
activating the light source;
detecting, by the sensor, the intensity of the light of the test and reference channel SPE in the predetermined wavelength band, thereby detecting the presence of the target analyte.

33. The method of claim 32, further comprising choosing a binding constant of the capture molecules as a function of an expected target analyte concentration.

34. The method of claim 33, wherein the binding constant is less than ten times and more than one tenth of the expected target analyte concentration.

35. The method of claim 33, wherein the binding constant is less than a hundred times and more than one hundredth of the expected target analyte concentration.

36. The method of claim 32, where a concentration of the target analyte is calculated from the intensity of the light of the test and reference channel SPE in the predetermined wavelength band.
